(19) United States
(12) Patent Application Publication
(10) Pub. No.: US 2005/0277806 A1
(43) Pub. Date: Dec. 15, 2005

(54) Prosthetic implant for sub-urethral support, an instrument, an insertion kit, and a surgical method for implanting it

(76) Inventor: Bernard Gilbert Robert Cristalli, Soisy Sur Seine (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/001,897

(22) Filed: Dec. 1, 2004

(30) Foreign Application Priority Data
Jun. 10, 2004 (FR) FR-04 06300

(51) Int. Cl. A61F 2/02

(52) U.S. Cl. 600/30

(57) ABSTRACT

The invention provides a prosthetic implant for supporting the urethra, the implant comprising a flexible elongate element and being provided: with a central portion for being positioned beneath the urethra when the elongate element is in its functional position in the body; and two ends situated on either side of the central portion; wherein said ends are positioned relative to the central portion in such a manner that when the elongate element is in the functional position, one of the ends of the elongate element is disposed between the right internal obturator muscle and the right external obturator muscle, while the other end is disposed between the left internal obturator muscle and the left external obturator muscle. The prosthetic implant is suitable for correcting urinary incontinence.

### CROSS-REFERENCE TO RELATED APPLICATION

This application claims priority to co-pending French Application entitled, “A Prosthetic Implant for Sub-Urethal Support, an Instrument, an Insertion Kit, and a Surgical Method for Implanting It,” having Serial No. FR-04 06300, filed on Jun. 10, 2004, which is entirely incorporated herein by reference.

The present invention relates to the general technical field of implantable devices for correcting urinary incontinence, and in particular adapted to correcting effort urinary incontinence in women.

The present invention relates more particularly to a prosthetic implant for sub-urethral support comprising a substantially flexible elongate element and provided:
with a central portion for positioning under the urethra for support purposes when the elongate element is in its functional position in the body; and
two ends situated on either side of the central portion.

The invention also relates to a surgical instrument for implanting a prosthetic implant for sub-urethral support in a dissection space.

The invention also provides a kit for inserting a prosthetic implant for sub-urethral support.

The invention also provides a method of treating urinary incontinence in women in which the above-mentioned prosthetic implant is put into position in the body.

### BACKGROUND OF THE INVENTION

Urinary incontinence comprises involuntary loss of urine via the urethra. This involuntary loss generally comes from the fact that the tissue responsible for retaining urine is no longer capable of exerting sufficient force to oppose the forces expelling urine. Stress urinary incontinence corresponds in particular to a loss of urine that is not preceded by any sensation of need, and that takes place not while at rest, but while making an effort.

Among the surgical treatments proposed for correcting urinary incontinence, there exists the treatment involving urethral support by means of a tape.

The tape is put into place under tension beneath the middle portion of the urethra, and is intended to support the urethra during effort so as to prevent any leakage of urine. Several types of tape are known that are associated with different surgical approaches. There thus exists tension-free vaginal tape (TVT) associated with surgical treatment via the retro-pubic approach and trans-obturator tape (TOT) associated with surgical treatment via the trans-obturator approach.

For surgical treatment using the TVT tape, an incision is made in the vagina, beneath the urethra, and two incisions are made in the skin of the abdomen. The TVT tape is placed beneath the urethra using a specific instrument and then rises on either side of the bladder in order to be anchored in the abdominal wall.

Although effective, the method using the TVT tape is difficult to implement and presents non-negligible operative risks. There is a risk of the bladder being perforated while manipulating the needles used for putting the tape into place. More severe consequences have also been observed such as perforating the iliac artery or indeed the small intestine, leading to visceral complications that can be fatal. That is why it is essential with that method of surgery to perform a cystoscopy.

For surgical treatment using the TOT tape, incisions are made firstly in the vaginal wall and secondly in the inside face of each thigh facing the obturator holes. The TOT tape used in the context of that method has a central portion placed beneath the urethra and designed to pass through the obturator holes and subsequently come to be anchored via its ends in the groin. Although that method presents a lower risk of perforating vital organs than does the method by the retro-pubic approach, it nevertheless does not completely eliminate the risk of vascular complications because of the length of the path occupied by the tape within the body.

### OBJECTS AND SUMMARY OF THE INVENTION

Consequently, the objects of the invention are to provide a novel prosthetic implant for sub-urethral support that does not include the above-mentioned drawbacks and that enables effort urinary incontinence to be corrected effectively while reducing the risks of operating complications.

Another object of the invention is to propose a novel prosthetic implant making it possible for surgery to be simple, with minimum risk, and via the vaginal approach exclusively.

Another object of the invention is to propose a novel prosthetic implant suitable for being put into place and held substantially stationary without tension.

Another object of the invention is to propose a novel prosthetic implant which is particularly easy to put into place.

Another object of the invention is to propose a novel prosthetic implant that is simple and reproducible to fabricate.

The objects of the invention also include providing a novel surgical instrument and a novel insertion kit for implanting a prosthetic implant for sub-urethral support enabling an intervention to be carried out that is simple and easily reproducible.

Another object of the invention is to provide a-novel surgical instrument and a novel insertion kit enabling the risk of operating error to be reduced.

Another object of the invention is to propose a novel surgical instrument and a novel insertion kit enabling the prosthetic implant to be put into place by the vaginal approach exclusively.

Another object of the invention is to propose a novel surgical instrument and a novel insertion kit enabling the intervention to be performed in outpatient surgery.

Another object of the invention is to propose a novel surgical instrument and a novel insertion kit that are particularly simple and intuitive to handle.

Another object of the invention is to propose a novel surgical instrument and a novel insertion kit that are particularly simple to fabricate and to shape.

The objects of the invention also include proposing a novel surgical method for correcting urinary incontinence in women, and enabling operating risks to be limited.

Another object of the invention is to provide a novel surgical method which is particularly simple and easily reproducible.

Another object of the invention is to propose a novel surgical method that can be performed via the vaginal approach exclusively.

Another object of the invention is to propose a novel surgical method that is suitable for being performed in outpatient surgery.

The objects of the invention are achieved by a prosthetic implant for sub-urethral support, the implant comprising an elongate element that is substantially flexible and provided:
with a central portion for positioning under the urethra for support purposes when the elongate element is in its functional position in the body; and
two ends situated on either side of the central portion;
wherein said ends are positioned relative to the central portion in such a manner that when the elongate element is in the functional position, its central portion then being substantially in register with the urethra, one of the ends of the elongate element is located between the right internal obturator muscle and the right external obturator muscle, with the other end being located between the left internal obturator muscle and the left external obturator muscle.

The objects of the invention are also achieved by a surgical instrument for implanting a prosthetic implant for sub-urethral support in a dissection space, the instrument comprising a substantially elongate guide support for being inserted into the dissection space, and specifically adapted so that said prosthetic implant can slide along the guide support towards its functional position in the body.

The objects of the invention are also achieved by a kit for inserting a prosthetic implant for sub-urethral support, the kit comprising:
a prosthetic implant for being inserted in a dissection space and for being positioned beneath the urethra in order to provide support therefor; and
a surgical instrument provided with a substantially elongate guide support, said guide support being designed to be inserted into said dissection space and being specifically adapted so that the prosthetic implant can slide along the guide support towards its functional position in the body.

The objects of the invention are also achieved by a surgical method for treating urinary incontinence in women, the method comprising a step c) of positioning a prosthetic implant comprising an elongate element provided with a central portion and two ends situated on either side of the central portion, during which step one of said ends is positioned between the right internal obturator muscle and the right external obturator muscle, and the other end is positioned between the left internal obturator muscle and the left external obturator muscle, in such a manner that said central portion is located substantially beneath the urethra, thus serving to support it.

### BRIEF DESCRIPTION OF THE DRAWINGS

Other advantages and objects of the invention will appear in greater detail on reading the following description and with the help of the accompanying drawings provided purely by way of non-limiting illustration, and in which:

FIG. 1 is a front section view showing a prosthetic implant for sub-urethral support in accordance with the invention, in its functional position in the body of the patient;

FIG. 2 is a plan view of an embodiment of the prosthetic implant in accordance with the invention;

FIG. 3 is a front view of the prosthetic implant shown in FIG. 2;

FIG. 4 is a plan view of the prosthetic implant shown in FIG. 2 in a state preceding its final shaping;

FIG. 5 is a front view showing an embodiment of a surgical instrument for implanting a prosthetic implant in accordance with the invention;

FIG. 6 is a plan view of the surgical instrument shown in FIG. 5; and

FIG. 7 is a section view on line A-A of FIG. 6 showing an embodiment detail of the surgical instrument shown in FIG. 6.

### MORE DETAILED DESCRIPTION

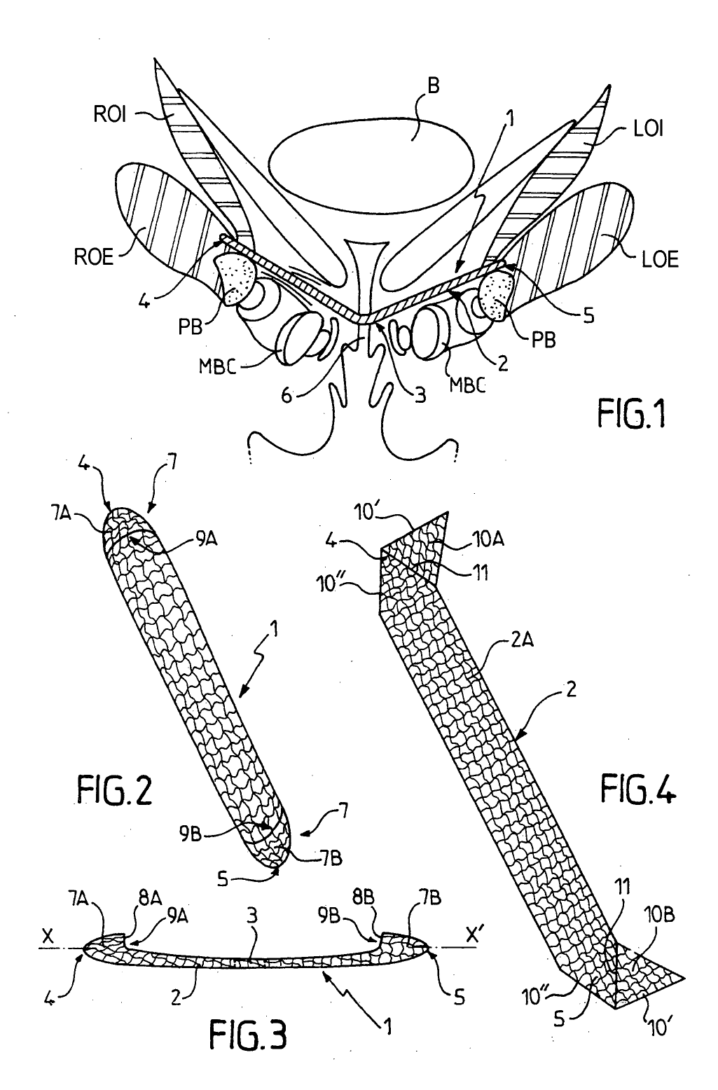

FIG. 1 shows a prosthetic implant in accordance with the invention for implanting in the body of a patient in order to provide support for the urethra in order to correct urinary incontinence, and in particular stress urinary incontinence in women.

The term “prosthetic implant” is used herein to mean a prefabricated prosthesis for inserting into the body of the patient. FIG. 1 shows the prosthetic implant 1 in its functional position, i.e. in the position in which it serves to reinforce the urethra and prevent involuntary leakage of urine.

In the invention, the prosthetic implant 1 comprises an elongate element 2, e.g. in the form of a textile tape, as shown in FIG. 2. The tape is preferably made of knitted polypropylene and advantageously presents an open structure, i.e. it is provided with openings defined by different sets of yarns. In the invention, the elongate element 2 is substantially flexible, i.e. it is sufficiently flexible firstly to avoid a phenomenon of shear in biological tissue after it has been put into place within the body, and secondly enabling it to take up an angled configuration as shown in FIG. 1. The elongate element 2 is also sufficiently flexible to be able to track any movements of the surrounding biological tissues once it has been implanted.

In the invention, the elongate element 2 comprises a central portion 3 for positioning beneath the urethra for support purposes when the elongate element 2 is in its functional position in the body, as shown in FIG. 1. This elongate element extends in a longitudinal direction X-X′ between two ends 4 and 5 that are situated on either side of the central portion 3. According to an essential characteristic of the invention, the ends 4 and 5 of the elongate element 2 are positioned relative to the central portion 3 in such a manner that when the elongate element 2 is in its functional position (FIG. 1), its central portion 3 is then substantially in register with the urethra in a conventional support configuration, with one of the ends (4) of the elongate element 2 being disposed between the right musculus obturator internus ROI and the right musculus obturator externus ROE, and the other end 5 being placed between the left musculus obturator internus LOI and the left musculus obturator externus LOE. Elsewhere in this specification, these muscles are referred to in English as the right and left, internal and external, obturator muscles.

The elongate element 2 is thus advantageously dimensioned in such a manner that when its central portion 3 is positioned beneath the urethra, its ends 4 and 5 can be positioned between the right and left internal and external obturator muscles firstly without putting the elongate element 2 under tension and secondly without releasing it excessively since that might lead to folds forming in its length.

The length of the elongate element 2 thus lies advantageously in the range 100 millimeters (mm) to 140 mm, and is preferably about 130 mm.

In particularly advantageous manner, the prosthetic implant 1 is merely put into place without tension, i.e. it does not require any positive anchor means penetrating into tissue for its placement. The prosthetic implant 1 thus advantageously constitutes a retro-obturator prosthesis that is merely put into place.

In addition, the elongate element 2 advantageously not does put the urethra under any tension, thus merely supporting the urethra and not suspending it. By cell invasion, scar tissue forms around the prosthetic implant 1, specifically around the elongate element 2, thus forming a matrix of scar tissue that is reinforced by the prosthetic implant, said matrix being capable of containing and opposing forces tending to expel urine. The open structure of the tape thus performs a role that is crucial insofar as it encourages scar tissue to form and thus achieve “natural” anchoring, i.e. without using any “positive” anchor means for the tape over its entire length.

The prosthetic implant 1 is advantageously adapted to be suitable for placement exclusively by the vaginal approach, by making a single incision in the vagina 6.

In order to ensure that the urethra is held effectively and comfortably while minimizing risks of rejection, the elongate element 2 advantageously presents a length lying substantially in the range 10 mm to 20 mm, and preferably of about 15 mm.

In a particularly advantageous embodiment, which in itself constitutes an invention, the elongate element 2 is provided at its ends with non-penetrating holding means 7 for holding it in position (FIG. 2). The holding means 7 act advantageously merely by pressing and rubbing against biological tissue and without penetrating therein, and serve to ensure that the elongate element 2 is prevented from moving sufficiently to enable cells to invade around the prosthetic implant 1. Thus, although the formation of scar tissue is generally rapid, it can happen that excessive play for the prosthetic implant has the effect of significantly increasing the time required for scar formation. The holding means 7 thus enable the play of the tape to be limited while nevertheless allowing the tape, because of its flexibility, to track any small amplitude movements of the surrounding tissues.

In particularly advantageous manner, the holding means 7 are formed by a structure that is sufficiently flexible to be capable of bending while the prosthetic implant 1 is being put into place, and of deploying once said implant is in its functional position. Thus, during placement of the implant and while it is being moved in the dissection space, it is particularly advantageous to ensure that the implant does not oppose such movements, for example by catching on biological tissue. In contrast, once the prosthetic implant 1 is in place in its functional position, deployment of the holding means 7 enables the elongate element 2 to be held in position, encouraging contact with the surrounding biological tissues.

In a preferred embodiment of the invention as shown in FIGS. 2 and 3, the holding means 7 are preferably formed by pockets 7A and 7B. As shown in FIG. 3, in the deployed position, the pockets 7A and 7B present respective retaining rims 8A, 8B suitable for constituting additional obstacles against movement of the elongate element 2. The pockets 7A, 7B thus serve individually to hold the ends 4, 5 of the elongate element 2 in position relative to the surrounding biological tissues.

In even more preferred manner, the pockets 7A, 7B are mounted in opposition at the ends 4, 5 of the elongate element 2 so as to prevent the implant from moving by acting mutually. Thus, since the retaining rims 8A, 8B are placed facing each other, as can be seen in FIG. 2, they prevent the elongate element 2 from moving in the longitudinal direction X-X′.

In particularly advantageous manner, the pockets 7A, 7B open beside the central portion 3 of the elongate element 2 via inlets 9A, 9B suitable for receiving a placement tool (not shown) for putting the prosthetic implant 1 into place and provided with a contact member such as a curved scissor tip that is designed to press against the prosthetic implant 1. Thus, the pockets 7A, 7B can act as means for receiving the placement tool, and within which said placement tool and more specifically its contact member, e.g. the rounded scissor tip, can come to bear in order to push the prosthetic implant 1 through the dissection space, thus making it easier to put into place.

The elongate element 2 and the pockets 7A and 7B are preferably made from the same textile tape. Thus, as shown in FIG. 4, the elongate element 2 is advantageously provided at its ends 4, 5 with two flaps 10A, 10B suitable for being folded along a fold line 11 so as to form the pockets 7A, 7B. Preferably, the flaps 10A, 10B are folded in such a manner that one of the edges 10′ of the flaps 10A, 10B overlies the corresponding edge 10″ of the main portion 2A of the elongate element 2 so as to be sewn thereto (FIG. 4). This technique for making the pockets is naturally given purely by way of illustration and other configurations could equally well be devised without going beyond the ambit of the invention.

According to a particularly advantageous characteristic of the invention, the pockets 7A, 7B are shaped to form respective tips, preferably rounded tips, so as to facilitate penetration and movement of the prosthetic implant 1 into the dissection space.

FIG. 5 shows a surgical instrument in accordance with the invention for implanting a prosthetic implant 1 for sub-urethral support in a dissection space. The dissection space is advantageously formed by an access path formed on either side of the urethra, oriented upwards, outwards, and forwards, beneath the vaginal wall, initially towards the ischio-pubic branch and then going round it from above, so as to allow the prosthetic implant 1 to pass therethrough.

In the meaning of the invention, the directions of the movements performed are specified relative to a person in the standing position. Thus “upwards” means towards the head, “downwards” means towards the feet, “inwards” means towards the inside of the body, “outwards” means towards the outside of the body, “forwards” means in the walking direction, and “rearwards” means in the opposite direction to walking.

According to an essential characteristic of the invention, the surgical instrument 20 comprises a substantially elongate guide support 21 for being inserted into the above-mentioned dissection space. According to the invention, the guide support 21 is specifically adapted so that the prosthetic implant 1 can slide along said guide support 21 towards its functional position in the body.

The guide support 21 thus advantageously presents a sliding surface 22. The prosthetic implant 1 is then designed to be moved into contact with said sliding surface 22 and to move therealong, the surface being used both as a support and as a guide.

Such a guide support 21 thus enables the surgeon to perform interventions that are easily reproducible, without risk of error.

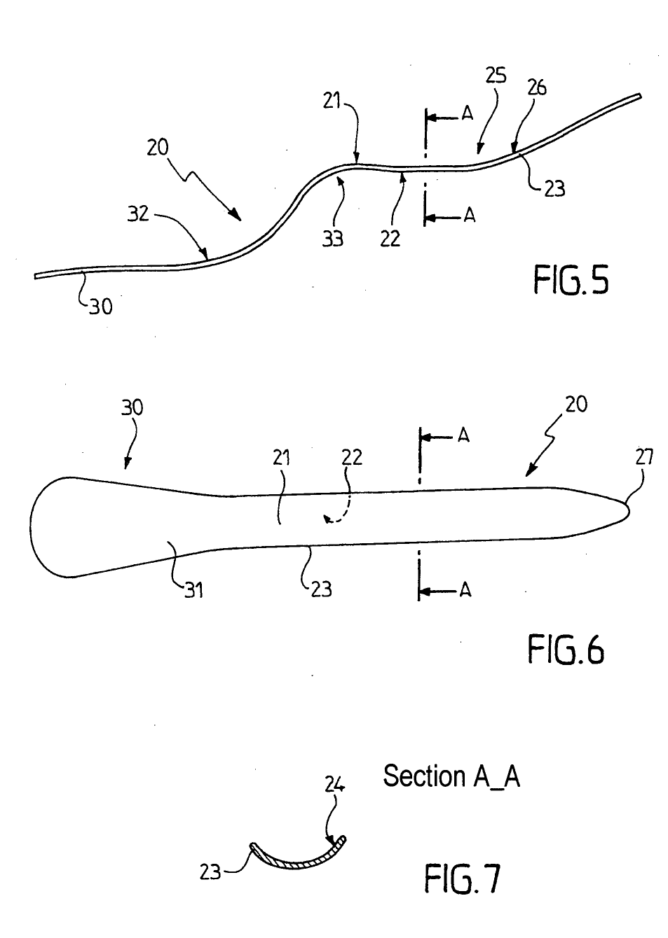

In particularly advantageous manner, and as shown in FIGS. 5 and 6, the guide support 21 is formed by a blade 23. In even more preferred manner, the blade 23 presents a U-shaped cross-section (FIG. 7) so as to form a slideway 24 for the prosthetic implant 1. This shape for the blade 23 thus makes it possible to further improve the guidance given to the prosthetic implant 1 towards its functional position. According to another advantageous aspect of the invention, the blade 23 is geometrically shaped in such a manner as to adapt to the anatomy of the dissection space, i.e. it can easily be inserted into the dissection space and it also fits substantially snugly against the tissues defining said space.

To this end, the blade 23 advantageously presents at least one first curved portion 25 along its length.

With the dissection space being defined downwards and outwards by the ischio-pubic branch, the first curved portion 25 advantageously presents curvature that is substantially complementary to the bone curvature of the ischio-pubic branch. In this way, the concave portion 26 of said first curved portion 25 fits substantially snugly around the convex internal structure of the ischio-pubic branch. The surgical instrument 20 then advantageously constitutes an anatomic applicator enabling the prosthetic implant 1 to be introduced along a path that is very accurate, situated in the direct proximity of the surgical instrument, and thus without risk of damaging surrounding tissues.

In a particularly advantageous aspect of the invention, the surgical instrument 20 advantageously includes a handle portion 30 formed by an extension 31 of the blade 23 so as to make the instrument easier to handle by the surgeon, the handle portion 30 preferably being substantially wider than the blade 23. Thus, when implementing surgical techniques minimizing invasion, it is preferable to restrict the dimensions of the active portion of the instrument, i.e. the blade 23, while nevertheless making the surgical act easier. To this end, the handle portion 30 advantageously presents longitudinal curvature to make it easier to insert the guide support 21, specifically the blade 23, into the dissection space. The handle portion 30 then forms a second curved portion 32 (FIG. 5). The surgical instrument 20 thus presents two curves, giving it an improved anatomic nature.

Preferably, and as shown in FIG. 5, the first and second curved portions 25 and 32 both present curvature oriented in the same direction.

Advantageously, the blade 23 includes a third curved portion 33 situated between the first and second curved portions 25 and 32, and presenting curvature that is inverted relative to said first and second curved portions. This third curved portion 33 thus advantageously makes it possible to go around biological tissues while inserting the surgical instrument 20 into the dissection space.

In order to encourage penetration and displacement of the surgical instrument 20 in the dissection space, while being atraumatic for the biological tissue, the leading edge 27 of the blade 23 is in the form of a blunt tip (FIG. 6). Preferably, the blade 23 is made of a material that is substantially rigid, for example a rigid polymer or a metal (stainless steel), however it is also possible to envisage using a blade 23 made of a material that is deformable and/or malleable in order to retain the possibility of adapting the anatomic shape of the instrument to each particular situation.

In preferred manner, the length of the surgical instrument 20 lies substantially in the range 120 mm to 180 mm, and is preferably about 160 mm.

The surgical instrument 20 thus comprises firstly the handle portion 30, of length preferably substantially equal to 50 mm, and secondly the guide support 21, advantageously formed by the blade 23, and of length that is preferably about 110 mm.

Furthermore, the optimum width of the guide support 21 is preferably about 15 mm, with the handle portion 30 preferably presenting greater width, of about 25 mm.

The present invention also provides a kit for inserting the prosthetic implant 1, the kit comprising firstly said prosthetic implant 1, and secondly the surgical instrument 20, as described above.

Advantageously, the insertion kit can also include the tool for placing the prosthetic implant 1 having characteristics as described above.

The surgical method for treatment of urinary incontinence in women in accordance with the invention is described below with reference to FIGS. 1 to 7.

To perform the intervention, the patient is installed in the gynecological position with the thighs hyperflexed.

The surgical method of the invention comprises initially an incision step a) during which there is made, under local anesthesia, loco-regional anesthesia, or indeed under general anesthesia, an incision in the anterior vaginal wall beneath the urinary meatus so as to enable the prosthetic implant 1 to be inserted. The incision is preferably a middle vertical incision that is about 15 mm to 30 mm long, and it is made about 15 mm beneath the urinary meatus.

The surgical method then advantageously includes a preparatory step b) during which the surgeon forms an access path on either side of the urethra beneath the vaginal wall, and more precisely the deep face of the vaginal wall using dissection scissors, said path leading towards the ischio-pubic branch, so as to allow the prosthetic implant 1 to be passed therealong.

Thus, starting from the incision and going towards the ischio-pubic branch, a first stroke is made on the right of the urethra and a second stroke is made on the left of the urethra (and vice versa), such that the first and second strokes form the access path. Substantially the same act is performed on either side of the urethra, i.e. a movement that is preferably upwards, outwards, and forwards towards the ischio-pubic branch so as to make the first and second strokes so that they are substantially symmetrical about the urethra. This movement is then continued over said bone branch while maintaining the same general direction so as to go round said bone branch.

This preparatory step b) thus makes it possible to avoid any risk of perforating vital organs, by initially forming accurately a well-defined access path that can be followed quite safely while putting the prosthetic implant 1 into place. The prosthetic implant 1 is thus not put into place blindly, unlike other surgical methods of the prior art.

The surgical method of the invention then comprises a positioning step c) in which each of the ends 4 and 5 of the prosthetic implant 1 is taken along the corresponding access path as previously prepared, with each of the ends 4 and 5 being taken to a position between the corresponding internal and external obturator muscles, optionally going through the obturator membrane, but without extending outside the obturator holes, i.e., in the meaning of the invention, without passing through the obturator holes, nor the plane of the skin.

Thus, one end 4 of the tape forming the prosthetic implant 1 is positioned between the right internal obturator muscle ROI and the right external obturator muscle ROE, with the other end 5 being placed between the left internal obturator muscle LOI and the left external obturator muscle LOC. This causes the tape to pass on either side of the urethra (not shown), above the musculus bulbo-cavernosus MBC above the ischio-pubic branch of the pubic bone PB, so as to position the ends of the tape behind the internal obturator muscle and above the deep aponeurosis.

During this step c) the central portion 3 of the elongate element 2 is naturally also positioned substantially beneath the urethra, in register therewith, so that said central portion 3 serves to support the urethra.

In particularly advantageous manner, when the tape is properly positioned, it is allowed merely to rest on the tissues of the dissection space without being fixed. It is thus cell invasion that serves to cause the tape to become progressively fixed within scar tissue.

In its functional position, the prosthetic implant 1 thus advantageously occupies a V-shape, with a wide flare angle so that the implant extends almost horizontally. There is thus no risk of the bladder B being perforated with the type of implant used in this method.

In particularly advantageous manner, the method of the invention also includes an intermediate step during which a surgical instrument 20 is inserted into the access path, said surgical instrument 20 comprising a guide support 21 against which the prosthetic implant 1 is subsequently caused to slide towards its functional position. The surgical instrument 20 thus advantageously constitutes an anatomic applicator, designed to be slid successively into each of the two strokes made on either side of the urethra.

During insertion, the concave portion 26 of the first curved portion 25 of the surgical instrument 20 faces downwards and outwards and turns about the ischio-pubic branch, following its convex shape. The surgeon subsequently proceeds with placing the prosthetic implant 1 which is performed starting from the incision already made in the vaginal wall, by pushing the implant towards its final or functional position along the slideway 24 formed by the blade 23, i.e. without it being necessary to pull on said prosthetic implant 1 from an additional incision made at any other location in the body of the patient.

To this end, the surgeon preferably uses an object (or placement tool) presenting a tapering end, such as the tip of rounded scissors, in order to take hold of the prosthetic implant 1. In particular, the tip of the scissors is inserted into one of the pockets 7A, 7B located at the ends 4, 5 of the prosthetic implant 1, e.g. a polypropylene tape. The scissor tip is then inserted into one of the two strokes, along which it is guided by the chute formed by the applicator, thus bringing the corresponding end of the tape into its final position. Once the tape has been correctly positioned, it is held in position by the scissor tip while the applicator is being withdrawn. The scissors are withdrawn only subsequently, thus leaving half of the tape in the stroke. A substantially identical action is reproduced on the other side of the urethra so as to position the second half of the tape.

Following this procedure, the prosthetic implant 1 is merely put into place, and it is not suspended, nor is it secured to the biological tissue by any fixing means.

Following implantation of the prosthesis, it is advantageously the holding means 7 which serve to hold the prosthesis sufficiently steady to enable cell invasion to take place quickly.

Such a method presents the advantage of enabling intervention to be performed exclusively via the vaginal approach, without it being necessary to make additional incisions in the abdomen, the groin, the crural fold, or the internal faces of the buttocks in order to pull on the tape. This method thus uses the retro-obturator approach.

In addition, since the tape is particularly short, it makes it possible to limit any risk of puncturing vital organs and thus of avoiding additional inspections, such as cystoscopy. Unlike prior art techniques, there is no need for the tape to be fixed, and risks of rejection are also limited because the tape is short. The method of placing the tape is also particularly easy and rapid to implement and it is also advantageous from the point of view of its being innocuous. This method thus requires no perforation of parietal planes, which in association with the speed with which it can be performed, makes it possible very significantly to limit the risks of perforating vital organs or of consequential pain.

1. A prosthetic implant for sub-urethral support, the implant comprising an elongate element that is substantially flexible and provided:
with a central portion for positioning under the urethra for support purposes when the elongate element is in its functional position in the body; and
two ends situated on either side of the central portion;
wherein said ends are positioned relative to the central portion in such a manner that when the elongate element is in the functional position, its central portion then being substantially in register with the urethra, one of the ends of the elongate element is located between the right internal obturator muscle and the right external obturator muscle, with the other end being located between the left internal obturator muscle and the left external obturator muscle.

2. A prosthetic implant according to claim 1, wherein the elongate element is provided with holding means for holding it in position, said means being disposed at its ends and serving merely by pressing and rubbing against biological tissue, to hold the elongate element sufficiently steady to enable cellular invasion to take place around the implant.

3. A prosthetic implant according to claim 2, wherein the holding means are formed by a structure that is sufficiently flexible to fold while the implant is being put into place, and to deploy when said implant is in its functional position.

4. A prosthetic implant according to claim 2, wherein the holding means are formed by pockets mounted in opposition to each other at the ends of the elongate element.

5. A prosthetic implant according to claim 4, wherein the inlet to the pockets are situated beside the central portion of the elongate element.

6. A prosthetic implant according to claim 4, wherein the elongate element is provided with two flaps disposed at its ends and suitable for being folded so as to form the pockets.

7. A prosthetic implant according to claim 4, wherein the pockets are shaped to form respective tips so as to facilitate penetration and displacement of the implant in a dissection space.

8. A prosthetic implant according to claim 1, wherein the elongate element is made from a textile tape.

9. A prosthetic implant according to claim 8, wherein the tape is made of knitted polypropylene.

10. A prosthetic implant according to claim 1, wherein the length of the elongate element lies substantially in the range 100 mm to 140 mm, and is preferably about 130 mm.

11. A prosthetic implant according to claim 1, wherein the width of the elongate element lies substantially in the range 10 mm to 20 mm, and is preferably about 15 mm.

12. A surgical instrument for implanting a prosthetic implant for sub-urethral support in a dissection space, the instrument comprising a substantially elongate guide support for being inserted into the dissection space, and specifically adapted so that said prosthetic implant can slide along the guide support towards its functional position in the body.

13. An instrument according to claim 12, wherein the guide support is formed by a blade.

14. An instrument according to claim 13, wherein the blade presents a substantially U-shaped cross-section so as to form a slideway for the prosthetic implant.

15. An instrument according to claim 13, wherein the blade is geometrically shaped so as to match the anatomy of the dissection space.

16. An instrument according to claim 13, wherein the blade presents, longitudinally, at least one curved portion.

17. An instrument according to claim 16, wherein the dissection space is defined downwards and outwards by the ischio-pubic branch, and said first curved portion presents curvature that is substantially complementary to the bone curvature of the ischio-pubic branch so that said concave portion of said first curved portion fits substantially snugly against the convex internal surface of the ischio-pubic branch.

18. An instrument according to claim 13, including a handle portion formed by an extension of the blade.

19. An instrument according to claim 18, wherein the handle portion is substantially wider than the blade.

20. An instrument according to claim 18, wherein the handle portion presents longitudinal curvature for facilitating insertion of the guide support into the dissection space, thereby forming a second curved portion.

21. An instrument according to claim 16, wherein the first and second curved portions both present curvature oriented in the same direction.

22. An instrument according to claim 21, wherein the blade includes a third curved portion situated between the first and second curved portions and presenting curvature that is inverse relative to said first and second curved portions.

23. An instrument according to claim 13, wherein the leading edge of the blade is in the form of a blunt tip.

24. An instrument according to claim 13, wherein the blade is made of substantially rigid material.

25. An instrument according to claim 13, wherein the blade is made of malleable material.

26. An instrument according to claim 12, wherein its length lies substantially in the range 100 mm to 180 mm, and is preferably about 160 mm.

27. An instrument according to claim 12, wherein the length of the guide support is preferably about 110 mm.

28. A kit for inserting a prosthetic implant for sub-urethral support, the kit comprising:
a prosthetic implant for being inserted in a dissection space and for being positioned beneath the urethra in order to provide support therefor; and
a surgical instrument provided with a substantially elongate guide support, said guide support being designed to be inserted into said dissection space and being specifically adapted so that the prosthetic implant can slide along the guide support towards its functional position in the body.

29. A kit according to claim 28, including a tool for placing the prosthetic implant, the tool being provided with a contact member designed to come to bear against the prosthetic implant, and the prosthetic implant includes reception means for receiving the placement tool, within which said contact member is suitable for coming to bear in order to push the prosthetic implant along the guide support.

30. A kit according to claim 29, wherein the reception means are formed by pockets.

31. A kit according to claim 28, wherein the guide support is formed by a blade presenting, longitudinally, at least a first curved portion.

32. A kit according to claim 31, wherein the blade has first and second curved portions, both having curvature oriented in the same direction, together with a third curved portion situated between the first and second curved portions and of curvature that is inverse relative to said first and second curved portions.

33. A surgical method for treating urinary incontinence in women, the method comprising a step c) of positioning a prosthetic implant comprising an elongate element provided with a central portion and two ends situated on either side of the central portion, during which step one of said ends is positioned between the right internal obturator muscle and the right external obturator muscle, and the other end is positioned between the left internal obturator muscle and the left external obturator muscle, in such a manner that said central portion is located substantially beneath the urethra, thus serving to support it.

34. A method according to claim 33, including an incision step a), during which an incision is made in the vaginal wall so as to enable the prosthetic implant to be inserted.

35. A method according to claim 34, including, prior to the positioning step c), a preparation step b) during which an access path is formed on either side of the urethra from the incision and going towards the ischio-pubic branch so as to allow the prosthetic implant to pass.

36. A method according to claim 35, wherein the preparation step b) comprises a stage during which a first stroke is made from the incision towards the ischio-pubic branch to the right of the urethra, and a second stroke is made to the left of the urethra, said first and second strokes then together forming the access path for the prosthetic implant.

37. A method according to claim 36, wherein during the preparation step b), an upwards, outwards, and forwards movement is performed on either side of the urethra in order to make the first and second strokes such that said first and second strokes are substantially symmetrical about the urethra.

38. A method according to claim 37, wherein the movement for preparing the first and second strokes is continued above the ischio-pubic branch while substantially conserving the same general direction, so as to go around said ischio-pubic branch.

39. A method according to claim 1, wherein after positioning step c), the prosthetic implant is allowed to rest on biological tissue without being fixed thereto.

40. A method according to claim 35, wherein the positioning step c) comprises a stage of introducing a surgical instrument provided with a guide support into the access path, followed by a stage of guiding the prosthetic implant into position, during which stage the ends of the elongate element are taken along the access path while causing the prosthetic implant to slide along the guide support.

41. A method according to claim 40, wherein the stage of guiding into position is performed from said incision, with the prosthetic implant being pushed towards its final position within the body.

42. A method according to claim 34, wherein the incision step a) comprises a step during which a middle vertical incision is made beneath the urinary meatus.

43. A method according to claim 1, wherein during the positioning step c), the ends of the elongate element are brought between the internal and external obturator muscles without passing through the obturator holes.
